(19) United States
(12) Patent Application Publication
(10) Pub. No.: US 2011/0218497 A1
(43) Pub. Date: Sep. 8, 2011

(54) MULTIPLE-INJECTION MEDICAL APPARATUS

(76) Inventor: Antoine Assaf, Pianoro (IT)

(21) Appl. No.: 13/124,088

(22) PCT Filed: Oct. 14, 2009
(86) PCT No.: PCT/IB2009/054515

§ 371 Date: May 16, 2011

(30) Foreign Application Priority Data
Oct. 14, 2008 (IT) VI2008A00239

(51) Int. Cl. A61M 5/00

(52) U.S. Cl. 604/173

(57) ABSTRACT

A multiple-injection medical apparatus for injection of a medical fluid is described. The multiple-injection medical apparatus comprises a disk-like support member, a plurality of substantially radial needles housed in the disk-like support member, a chamber connected to one or more of the needles and suitable for containing the medical fluid to be injected, and a means for continuously refilling the chamber with the medical fluid after each injection.

### FIELD OF THE INVENTION

The present invention generally finds application to the field of equipment for medical and cosmetic use and particularly relates to a multiple-injection medical apparatus, particularly for subcutaneous, intradermal and intramuscular administration of a medical fluid for cosmetic surgery, allergy tests and similar techniques.

### BACKGROUND ART

Various procedures are known in medicine, in which medical fluids, such as bioactive substances, pharmaceuticals, medicaments, vaccines and the like, are intradermally or subcutaneously infused by injections performed in rapid succession on a part of the human body.

Particularly, in the field of cosmetic surgery, vitalizing agents, such as hyaluronic acid and amino acids, are injected into the dermis or subcutaneously using a syringe, to revitalize the skin by increasing hydration and promoting the formation of collagen.

This is currently done by manual procedures, which involve considerable difficulties even for expert physicians, as hundreds of contiguous injections have to be done in the part of the body to be treated.

It is very difficult to determine the optimal needle depth and the proper dose of fluid to be injected in each application, which involves wastes of injected substances, which are generally expensive.

A further problem encountered by the skilled person is that blood vessels under the skin should be avoided, to prevent the formation of undesired hematomas.

It should be also noted that the large number of successive injections to be made makes the whole procedure annoying and painful both for the patient, who is required to undergo an invasive treatment for a long time and for the physician, who may with time experience pain in the wrist.

Further procedures that require successive performance of a plurality of punctures or injections are known in the field of allergology, for allergy tests.

Also in this case, the operator manually does a number of punctures for introducing a medical substance contained in the needle or previously distributed over the part of the body being treated into the patient's skin, with the same drawbacks as mentioned above.

In an attempt to wholly or partially obviate the above drawbacks, a few multiple-injection medical devices have been provided, which could perform a multiplicity of punctures or injections of a medical substance, simultaneously or in rapid succession.

WO02/02180 discloses an apparatus for transdermal administration of a medicament which has a plurality of parallel needles, adapted to simultaneously act on the patient's skin in directions substantially perpendicular to the part of the body d to be treated.

In one embodiment, the needles have a hollow tubular passage connected to a medicament delivery chamber.

The needles are moved by means of a plunger which causes them to cover a stroke sufficient for skin penetration.

This prior art solution still has the advantage of not allowing proper control of the amount of liquid to be administered and the injection points.

Patent application US2007/073217 discloses a further apparatus for transdermal delivery of medical substances for aesthetic purposes, comprising a plurality of disks each having a number of peripheral needles designed for successively act on the skin as a result of the rotation of the disks.

The combination of rotation and pressure exerted by the physician facilitates successive penetration of needles which in turn cause mechanical transfer of the medical fluid in the skin.

While this prior art apparatus accelerates and simplifies the step of intradermal and subcutaneous delivery of the medical fluid, it is still required to be previously distributed on the patient's skin or on the outer surface of the needles.

It is clear that also in this case there is no way of correctly dosing the medical liquid and obtaining a uniform distribution all over the part of the human body being treated.

U.S. Pat. No. 6,475,160 discloses a device for carrying out allergy tests, which also has a rotating disk with a plurality of needles arranged along its periphery and designed to penetrate the skin upon rotation of the disk.

The medical substance to be injected is contained in a plurality of radial inner conduits associated with respective needles to be injected as the corresponding needle is in contact with the skin.

While this solution obviates the problem of proper dosing of the medical substance to be injected within each conduit, it still does not ensure full transfer of the substance to the patient.

Therefore, proper operation of the device is still strictly dependent on the operators skill.

Furthermore, a relatively small amount of medical substance can be injected in each application and the device has to be replaced or refilled after each turn of the disk, which strongly limits the operativity of the device.

Also importantly, before reuse, the device must be disassembled to refill its conduits with the substance, which involves apparent operating complications, that make it difficulty reusable.

### DISCLOSURE OF THE INVENTION

The object of the present invention is to overcome the above drawbacks, by providing a multiple-injection medical apparatus that is highly efficient and simple to use.

A particular object is to provide a multiple-injection medical apparatus that allows a large number of injections to be done with a relatively limited number of needles, in short times and with reduced stress both for the patient being treated and for the operator that uses it.

A further object is to provide a medical apparatus that allows proper dosing of the fluid to be injected by each puncture, thereby limiting wastes and providing considerable cost advantages.

A further object is to provide a medical apparatus that can be simply and quickly refilled with a new amount of medical fluid after each cycle of use.

A further object is to provide a medical apparatus that allows simple and quick replacement of used needles, or sterilization for reuse.

These and other objects, as more clearly shown hereafter, are fulfilled by a multiple-injection medical apparatus as defined in claim 1, which comprises a disk-like support member with a lateral surface defining a substantially central axis of rotation and designed to come into contact with the skin of a patient, a plurality of substantially radial needles housed in said disk-like member in angularly offset positions and radially project out of the side surface to selectively act on the patient's skin in response to the rolling of said disk-like member to inject predetermined doses of fluid, at least one chamber associated with one or more of said needles, for containing a predetermined amount of a medical fluid to be injected.

The apparatus is characterized in that it comprises means for continuously feeding medical fluid, which are susceptible to refill said chamber with a new predetermined amount of medical fluid after each injection, to allow each of said needles to perform a multiplicity of injections during the rolling of the disk-like member.

With this particular feature of the invention, the apparatus will be able to continuously perform a relatively large number of injections with a small number of needles or similar injection devices by simple rotation of the disk-like support member.

Thus, the operator will not have to perform a multiplicity of successive injections, but one quick step will be only needed, which will reduce the time required for the whole operation and the resulting stress for the patient.

Advantageously, the continuous feeding means may include a syringe removably connected to the feeding conduit and defining a reservoir for the medical fluid to be injected.

With this additional feature of the invention the apparatus will be able to control the total amount of fluid to be injected for each treatment, and will also allow reuse of the needles for multiple operations, after sterilization, by replacing the syringe only.

### BRIEF DESCRIPTION OF THE DRAWINGS

Further features and advantages of the invention will be more readily apparent upon reading of the detailed description of a preferred non exclusive embodiment of a multiple-injection medical apparatus of the invention, which is shown as a non limiting example with the help of the annexed figures, in which:

FIG. 1 is a perspective view of an apparatus of the invention in a first preferred configuration;

FIG. 2 is a front view of the apparatus of FIG. 1;

FIG. 3 is a side view of the apparatus of FIG. 1;

FIG. 4 is an exploded perspective view of the apparatus of FIG. 1;

FIG. 5 is an exploded perspective view of a detail of the apparatus of FIG. 1;

FIG. 6 is a first partial cross-sectional view of the detail of FIG. 5;

FIG. 7 is a second partial cross-sectional view of the detail of FIG. 5;

FIG. 8 is a perspective view of an apparatus of the invention in a second preferred configuration;

FIG. 9 is an exploded perspective view of the apparatus of FIG. 8.

### DETAILED DESCRIPTION OF ONE PREFERRED EMBODIMENT

Referring to the above figures, the apparatus of the invention, generally designated by numeral 1, may be used for performing a multiplicity of successive injections in one or more parts of a human body.

Particularly, the apparatus may be used in cosmetic surgery treatments, allergy tests and in all medical treatments that require transdermal or subcutaneous injection of one or more medical or therapeutic substances.

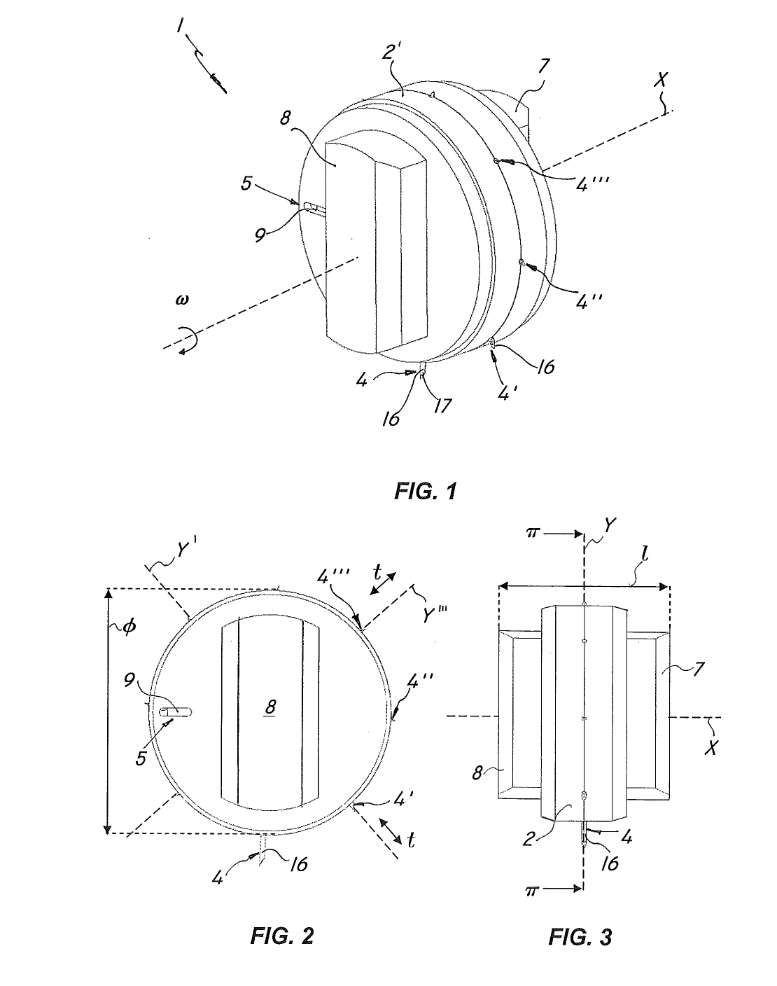

As shown in FIGS. 1 to 4, the multiple-injection medical apparatus of the invention comprises, in its basic configuration, a support disk-like member 2 with a lateral surface 2′ defining a substantially central axis of rotation X and designed to come into contact with the skin of a patient, at least one chamber 3 for containing a predetermined amount of a medical fluid to be injected and a plurality of substantially radial needles 4, 4′, 4″, . . . in fluid communication with the chamber 3.

The needles 4, 4′, 4″, . . . are housed in the disk-like member 2 in substantially coplanar and angularly offset positions and radially project out of the side surface 2′ thereof, to selectively act on the patient's skin in response to the rolling ω of the disk-like member 2 on the skin, thereby injecting at least part of the fluid that is fed thereto.

According to a peculiar feature of the invention, the apparatus 1 comprises means 5 for continuously feeding the chamber 3 with a controlled and predetermined amount of medical fluid, thereby refilling the needles 4, 4′, 4″, . . . with a new amount of fluid after each injection, to allow each needle 4, 4′, 4″, . . . to perform a plurality of injections within one operating cycle.

The needles 4, 4′, 4″, . . . will be referenced hereinafter by a single non-indexed numeral 4, all the parts in one of the needles 4 being intended to be also found in a substantially similar and functionally equivalent manner in the others.

In the illustrated configuration, the disk-like member 2 has a substantially cylindrical shape and is equipped with a single series of angularly equally spaced needles 4 arranged over a single diametral plane π substantially orthogonal to the axis of rotation X and substantially coincident with the median plane of the disk-like member 2.

However, an alternative embodiment, not shown, may provide additional series of needles 4 arranged over parallel and axially offset planes. Furthermore, the disk-like member 2 may also have a non-cylindrical shape, such as a conical, frustoconical shape or the like.

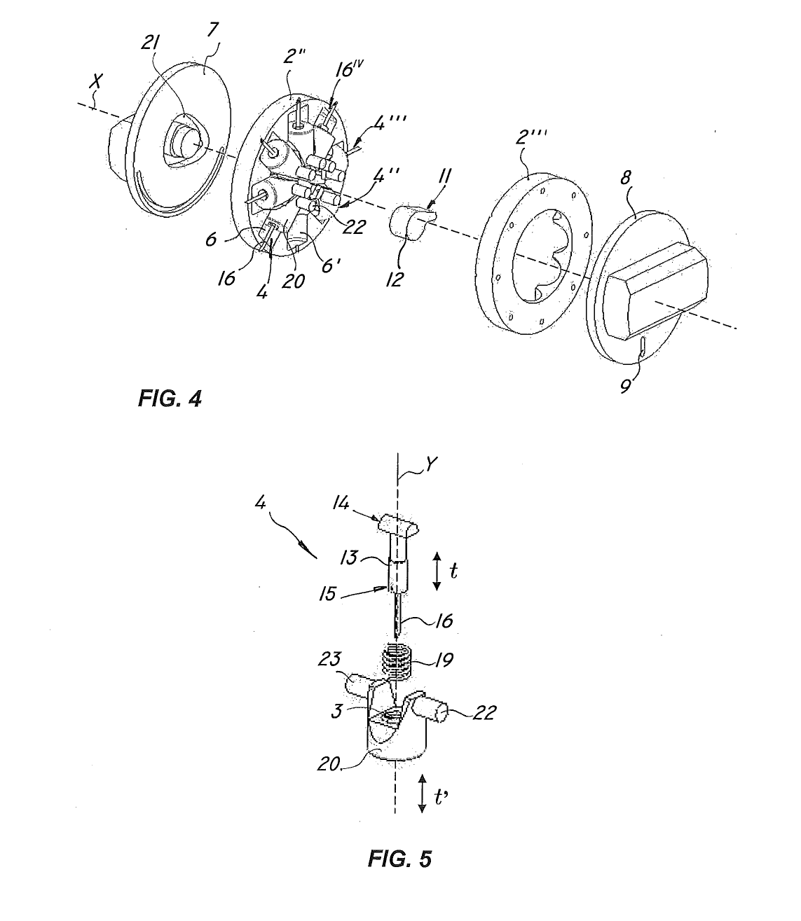

As more clearly shown in the exploded view of FIG. 4, the disk-like member 2 may be composed of a pair of cylindrical portions 2″, 2″ having mating inner shapes, to define a plurality of housings 6, 6′, 6″, . . . for corresponding needles 4.

Furthermore, the disk-like member 2 may be enclosed between two end shell halves 7, 8, which are preferably stationary with respect to the disk-like member 2 and may also act as a grip member for simple manual handling of the apparatus 1 by an operator.

The assembly composed of the disk-like member 2 and the shell halves 7, 8 may have a very small size. As an example, the maximum axial dimension 1 may be of 2 mm to 100 mm and the maximum diameter φ may be of 8 mm to 100 mm.

Thus, the disk-like member 2 may house 100 or more needles 4 arranged over a multiplicity of different planes, e.g. 30 planes or more, for performing hundreds of injections in rapid succession.

Advantageously, the continuous feed means 5 may include at least one feeding conduit 9 for feeding the chamber 3, the conduit 9 being designed to be connected to an external reservoir containing the fluid to be injected.

Particularly, a single feeding conduit 9 may be used for serving a plurality of fluid containing chambers 3, 3′, 3″, . . . associated with corresponding needles 4.

For clarity and simplicity the chambers 3, 3′, 3″, . . . and their parts will be also referenced hereinafter by non-indexed numerals.

Each chamber 3 may have a substantially cylindrical shape, which is defined by the particular shape of the housings 6 of the cylindrical portions 2″, 2′″ of the disk-like member 2.

Furthermore, each chamber 3 may have a fluid receiving hole 10, which is designed to be selectively connected to the common feeding conduit 9 upon rotation ω of the disk-like member 2.

The latter may also have means 11 for radially and successively moving the needles 4, which are designed to selectively and successively turn the rotary motion ω of the disk-like member 2 about its center axis X into a radial unidirectional or bidirectional motion t of each needle 4.

Advantageously, the radially moving means 11 may include a first cam body 12 rotatably mounted about the axis of rotation X to selectively interact with the needles 4 and cause projection thereof from the disk-like member 2 in corresponding radial directions Y.

As more clearly shown in the exploded view of FIG. 5, each needle 4 may have a substantially cylindrical cam-follower 13 with an inner radial end 14 facing towards the first cam body 12 and an outer radial end 15 equipped with a pointed injection cannula 16.

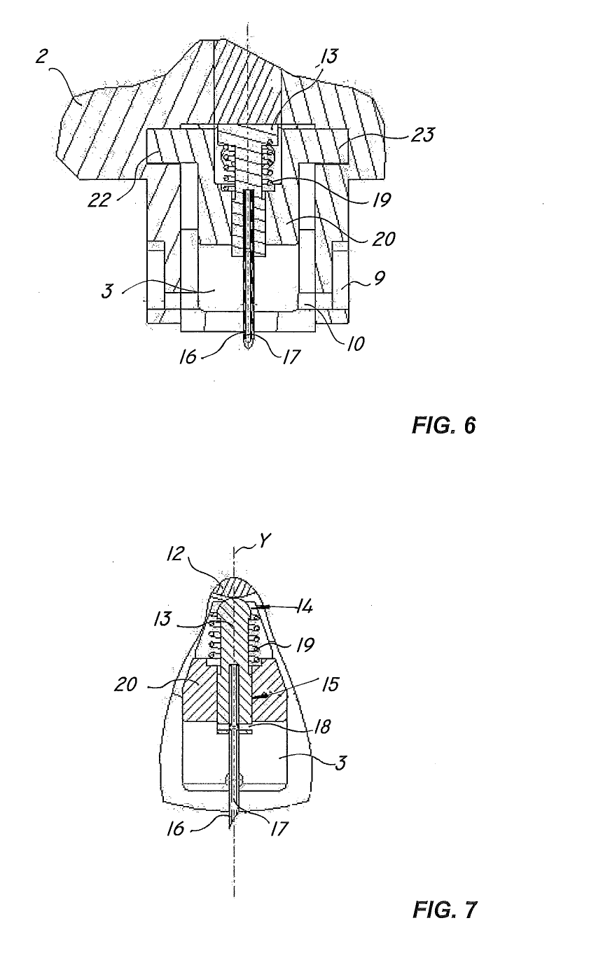

As shown in the cross sections of FIG. 6 and FIG. 7, the injection cannula 16 may have a radial tubular cavity 17 and each of the cam-followers 13 may have an opening 18 for fluid connection of its respective tubular cavity 17 with a corresponding chamber 3.

Thus, the medical fluid may be ejected from the chamber 3 for the transdermal or subcutaneous injection.

The cannulae 16 may have such a size as to radially project out of the disk-like member 2 to a predetermined length, according to the type of injection to be done.

For example, for transdermal injections, the cannulae 16 may radially project through a length of 0.1 mm to 4 mm, whereas the diameter of the tubular cavities 17 may be of 0.05 mm to 1 mm, according to the amount of fluid to be injected by each step.

The cylindrical chambers 3 may have an inside diameter of 0.5 mm to 30 mm to contain an amount of fluid from 0.001 ml to 1 ml.

Nevertheless, such dimensions shall be merely intended by way of example, the sizes of the cannulae 16 being selected according to the particular types of treatment to be performed.

Furthermore, each cam-follower 13 may be equipped with an elastic return element 19, such as a helical spring coaxial with the cam-follower 13 and operating on a corresponding cannula 16.

The elastic element 19 will force the radial return stroke t of the cannula 16 upon injection of the fluid and after disengagement of the first cam body 12 from its respective cam-follower 12 in response to the rolling ω of the disk-like member 2 through a predetermined angle.

Conveniently, the continuous feed means 5 may include, for each of the chambers 3, a plunger 20 capable of radially sliding in the corresponding chamber 3 to force the fluid into the cannula 16.

Furthermore, the moving means 11 may include at least one second cam body 21 selectively acting on the plungers 20 to turn the rotary motion ω of the disk-like member 2 into the radial sliding motion t′ of the plungers 20.

Particularly, the moving means 11 may include a pair of second cam bodies 21, one of which is only shown, which are integral with their respective shell halves 7, 8.

In the illustrated configuration, the second cam bodies 21 may be desmodromic cams for controlling the stroke of the plungers 20 in both directions in their respective radial sliding directions Y.

For instance, the second cam bodies 21 may be defined by specially shaped guides formed in each of the shell halves 7, 8 at a center position thereof, and substantially in mirror relation to each other, with respect to the median plane π of the disk-like member 2.

Each plunger 20 may have a pair of projections 22, 23 arranged symmetrically with respect to the radial direction Y of the corresponding needle 4 to engage a corresponding guide 21.

The first and second cam bodies 12, 21 may be suitably arranged for synchronized motion of each needle 4 with its plunger 20.

Particularly, the cam bodies 12, 21 may cause each plunger 20 to only move down into its chamber 3 when the opening 18 of the cam-follower 13 is not obstructed but in a position that provides fluid communication of the cannula 16 with the chamber 3, as shown in FIG. 7.

Furthermore, the upward movement of the plungers 20 will create a slight negative pressure condition in the chambers 3, and facilitate filling thereof with a new amount of fluid.

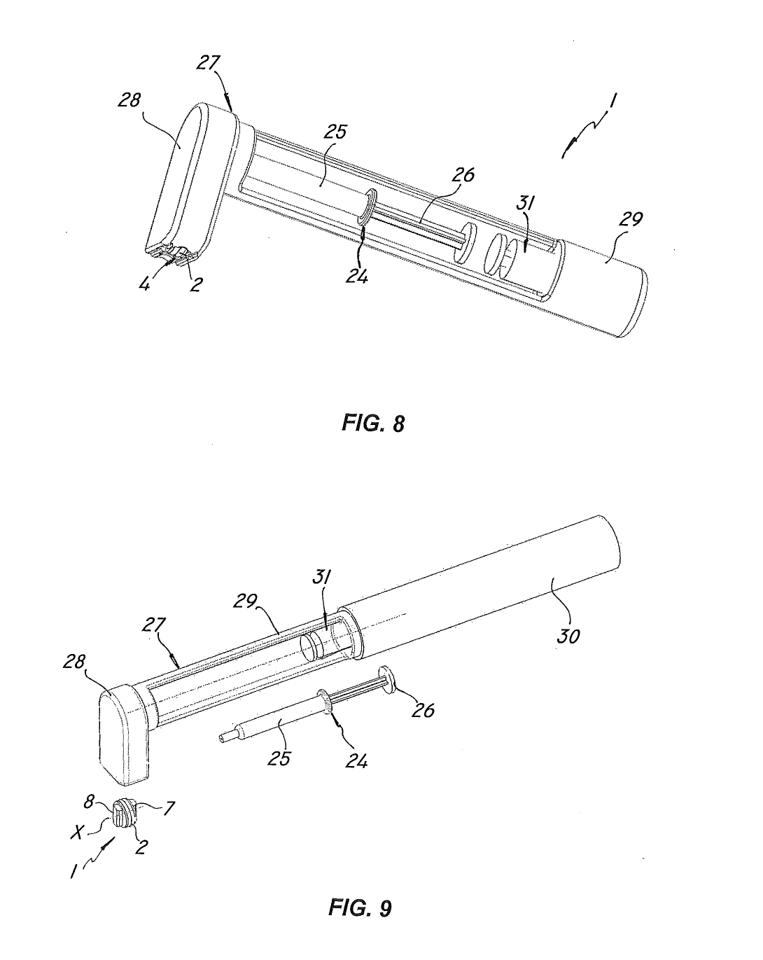

In a further embodiment of the invention, as shown in FIGS. 8 and 9, the continuous feed means 5 may include a syringe 24 defining a reservoir for the fluid to be injected.

The syringe 24 may be of common, preferably but without limitation disposable type, i.e. formed of a cylindrical sleeve 25 adapted to contain the fluid and designed to be connected to the feeding conduit 9, and a plunger 26 capable of sliding in the sleeve 25 to allow fluid into the feeding conduit 9.

The connection between the syringe 24 and the feeding conduit 9 may be provided by a small tube, not shown, whose length and inside diameter are selected according to the size of the syringe 24 and the feeding conduit 9.

Advantageously, an enclosure 27 may be provided, with a front portion 28 adapted to contain the disk-like member 2 and the needles 4, and a back elongate portion 29 of a size adapted to contain the syringe 24.

The front portion 28 is partially open, to allow the cannulae 16 to come out and interact with the patient's skin, as shown in FIG. 8, whereas the back portion 28 may have a removable cover 30 allowing access to its interior.

Actuator means 31 may be also provided, such as an electronically controllable linear pushing actuator, which operate on the plunger 26 of the syringe 24 and cause it to move at controlled speed and pressure within the sleeve 25 and control injection of the medical fluid.

Thus, no particular skill will be required of the operator, the amount of fluid injected by each needle 4 in each injection being controlled by simply calibrating the speed of the plunger 26.

The apparatus 1 may be also used without being connected to any fluid-containing reservoir or syringe, and hence without fluid, for instance in cosmetic surgery procedures aimed at stimulating the skin to collagen formation by a plurality of punctures.

Heat sterilizable or cold sterilizable materials or disposable materials may be used, according to the particular application.

For example and without limitation, the materials for an apparatus 1 of the type as shown in FIG. 1 may be engineering polymers, ceramic or metal materials, silicone rubbers and the like.

The above disclosure clearly shows that the invention fulfils the intended objects and particularly meets the requirement of providing a multiple-injection medical apparatus that can perform a multiplicity of injections in rapid succession with a relatively small number of needles.

The particular configuration of the medical fluid feeding means will allow very simple dosing of very small amounts of fluid to be injected in each injection.

The apparatus of the invention is susceptible of a many changes and variants within the inventive principle disclosed in the annexed claims. All the details thereof may be replaced by other technically equivalent parts, and the materials may vary depending on different needs, without departure from the scope of the invention.

While the apparatus has been described with particular reference to the annexed figures, the numerals referred to in the disclosure and claims are only used for the sake of a better intelligibility of the invention and shall not be intended to limit the claimed scope in any manner.

1. An apparatus for transdermal or subcutaneous injection of a medical fluid, wherein the apparatus comprises:
a) disk-like support member with a lateral surface defining a substantially central axis of rotation and designed to come into contact with skin of a patient;
b) a plurality of substantially radial needles housed in said disk-like support member in angularly offset positions to radially project out of the disk-like support member, wherein the plurality of substantially radial needles is configured for injecting set doses of the medical fluid and to selectively act on the patient's skin in response to rolling of said disk-like support member on the patient's skin;
c) at least one chamber suitable for containing a set amount of the medical fluid to be injected, said at least one chamber being associated to one or more substantially radial needles of said plurality of substantially radial needles; and
d) means for continuous feeding of the medical fluid, wherein the continuous feeding means is configured to refill said at least one chamber with a new amount of the medical fluid after each injection of the medical fluid in such a manner to allow said plurality of substantially radial needles to perform a multiplicity of injections during the rolling of said disk-like support member.

2. The apparatus according to claim 1, wherein said continuous feeding means comprises at least one feeding conduit for feeding the medical fluid to said at least one chamber, and wherein said at least one feeding conduit is connectable to an external reservoir containing the medical fluid to be injected.

3. The apparatus according to claim 2, wherein the at least one chamber is a plurality of chambers connected to respective substantially radial needles of the plurality of substantially radial needles, and wherein each of said chambers is provided with a through hole connected to said at least one feeding conduit for fluid entry.

4. The apparatus according to claim 1, wherein said disk-like support member comprises means for radially moving said plurality of substantially radial needles.

5. The apparatus according to claim 4, wherein said radially moving means comprises a first cam body rotatably mounted on said substantially central axis of rotation to selectively interact with said plurality of substantially radial needles and to promote projection thereof from said disk-like support member along respective radial directions.

6. The apparatus according to claim 5, wherein each of said plurality of substantially radial needles has a substantially cylindrical cam-follower with an internal radial end facing said first cam body and an external radial end provided with an injection cannula.

7. The apparatus according to claim 6, wherein said injection cannula has a radial hollow cavity, and wherein said substantially cylindrical cam-follower has an inlet for fluidic connection of said radial hollow cavity to a respective chamber to allow ejection of the medical fluid.

8. The apparatus according to claim 6, wherein each of said substantially cylindrical cam-followers is provided with an elastic return member acting on a respective substantially radial needle to force radial translation of the respective substantially radial needle toward an internal side of said disk-like support member after injection of the medical fluid.

9. The apparatus according to claim 6, wherein said continuous feeding means comprises, for each of said chambers, a plunger radially sliding in a respective chamber to force the medical fluid into said injection cannula.

10. The apparatus according to claim 9, wherein said radially moving means comprises at least one second cam body selectively acting on said plungers to transform the rolling of said disk-like support member into a radial sliding motion of said plungers.

11. The apparatus according to claim 2, wherein said continuous feeding means comprises a syringe with a cylindrical jacket adapted to contain the medical fluid to be injected and designed to be connected to said at least one feeding conduit and a pushing rod suitable for sliding into said cylindrical jacket to allow flowing of the medical fluid toward said at least one feeding conduit.

12. The apparatus according to claim 11, further comprising a casing with a front portion adapted to house said disk-like support member and said plurality of substantially radial needles and a back portion adapted to house said syringe.

13. The apparatus according to claim 11, further comprising actuating means acting against said pushing rod of said syringe for controlling translation of the pushing rod into said cylindrical jacket and regulating injection of the medical fluid.
